(19) United States
(12) Patent Application Publication
(10) Pub. No.: US 2007/0039624 A1
(43) Pub. Date: Feb. 22, 2007

(54) Patient compliance system and method to promote patient compliance

(76) Inventors: Richard H. Roberts, Lakewood, NJ (US); Kristin Arnold, Morrisville, PA (US)

Correspondence Address: CANTOR COLBURN, LLP, 55 GRIFFIN ROAD SOUTH, BLOOMFIELD, CT, 06002, US

(21) Appl. No.: 11/207,077

(22) Filed: Aug. 18, 2005

(51) Int. Cl. A61B 19/00; G06F 19/00; G06Q 10/00

(52) U.S. Cl. 128/898; 705/3

(57) ABSTRACT

Disclosed herein are systems and methods to promote patient compliance with a dosage regimen of a prescribed pharmaceutical agent.

### BACKGROUND

The effectiveness of a particular dosage regimen of a pharmaceutical to treat a disease or disorder oftentimes depends upon the patient's strict adherence to the particular regimen. Such a regimen can require the patient to take the medication according to set time intervals for days, weeks, or longer. Furthermore, many regimens are most effective if the patient takes all of the doses prescribed to them. Missed doses or failure to complete the full regimen may result in a recurrence of the disease or disorder, continued illness, ineffectiveness of future administration of the pharmaceutical, for example bacterial resistance as a result of poorly adhered to antibiotic regimens; continued illness; increased health costs in terms of both time and money; etc.

Non-compliance, especially among pediatric and geriatric patients, can be due to a number of factors including a sense of feeling better and subsequent refusal to continue with the regimen, lack of desire to continue the regimen especially if the regimen inconveniences the patient or if the medication has unpleasant side effects, a bad taste, or makes the patient feel ill, or even a general lack of motivation on the part of the patient.

Patient caregivers caring for a patient who refuses to take or is difficult about taking their medication have to expend more time and effort for the patient to follow a particular dosage regimen.

Patient compliance of a dosage regimen will help to assure optimal benefits of the particular pharmaceutical, overall improved health of the patient, and fewer visits to the doctor, thereby reducing overall health costs for a particular individual.

There remains a need in the art for improving patient compliance with particular dosage regimens of pharmaceutical agents.

A particularly difficult problem is compliance with a dosing regimen in children. It is known that motivation tools can increase a child's compliance with almost any activity including grooming, schooling, etc. It would be helpful to have a motivation tool associated with medication in children.

### SUMMARY OF THE INVENTION

Disclosed herein are systems to promote patient compliance with a pharmaceutical dosage regimen comprising a documenting device to document administration to a patient of each dosage of a pharmaceutical agent according to a dosage regimen, or a motivation device to motivate a patient to adhere to a dosage regimen for a pharmaceutical agent, wherein the motivation device is optionally also a documenting device; and optionally further comprising an indicating device; wherein the patient or caregiver is rewarded for the patient completing the dosage regimen.

In another embodiment, a system to promote patient compliance comprises a documenting device to document administration to a patient of each dosage of a pharmaceutical agent according to a dosage regimen; and optionally further comprising an indicating device; wherein the patient or caregiver is rewarded for the patient completing the dosage regimen.

In another embodiment, a system to promote patient compliance with a pharmaceutical dosage regimen comprises a motivation device to motivate a patient to adhere to a dosage regimen for a pharmaceutical agent; wherein the motivation device is optionally also a documenting device to document administration to the patient of each dosage of the pharmaceutical agent according to the dosage regimen, optionally further comprising an indicating device; and wherein the patient or caregiver is optionally rewarded for the patient completing the dosage regimen.

In one embodiment, a method to promote patient compliance with a pharmaceutical dosage regimen comprises using a documenting device to document the administration to a patient of each dosage of a pharmaceutical agent according to a dosage regimen, or providing a motivation device to motivate a patient to adhere to a dosage regimen for the pharmaceutical agent, wherein the motivation device is optionally also a documenting device; optionally using an indicating device; and rewarding the patient or caregiver for the patient completing the dosage regimen as evidenced by the documentation.

In yet another embodiment a method to promote patient compliance with a pharmaceutical dosage regimen comprises using a documenting device to document the administration to a patient of each dosage of a pharmaceutical agent according to a dosage regimen; optionally using an indicating device; and rewarding the patient or caregiver for the patient completing the dosage regimen as evidenced by the documentation.

In still yet another embodiment, a method to promote patient compliance with a pharmaceutical dosage regimen comprises providing a motivation device to motivate a patient to adhere to a dosage regimen for a pharmaceutical agent; wherein the motivation device is optionally also a documenting device to document administration to the patient of each dosage according to the dosage regimen; and optionally rewarding the patient or caregiver for the patient completing the dosage regimen as evidenced by the documentation.

Also provided are kits to promote patient compliance with a pharmaceutical dosage regimen comprising a plurality of dosage units comprising a pharmaceutical agent; instructions of a dosage regimen for the pharmaceutical agent; and the systems described herein.

### BRIEF DESCRIPTION OF THE DRAWINGS

FIG. 1. An exemplary documenting device for documenting the successful administration of each dosage unit according to a dosage regimen.

### DETAILED DESCRIPTION OF THE INVENTION

Disclosed herein are systems and methods of promoting patient compliance with a pharmaceutical dosage regimen. Providing an incentive and/or a reward encourages patients, especially pediatric and geriatric patients, to complete their prescription according to a prescribed regimen. Increased compliance results in an increased likelihood that the disease or infection can be treated more completely.

The systems and methods use motivation devices, documenting devices, or a combination thereof. The systems and methods are not particularly limited as long as there is a way to provide an incentive and/or reward to the patient after administration of each dose or unit of doses of the pharmaceutical agent at the appropriate time in the regimen. Furthermore, the systems and methods optionally allow for the tracking and memorializing of the administration of each dose or unit of doses in a particular format.

As described herein, “unit of doses” can include any number of doses from a single dose, multiple doses, or all the doses of the regimen. The unit of doses may be described in terms of temporal limitations rather than numerically. For example, one unit can encompass all of the doses taken in a single day for BID or TID administration rather than tracking each single dose. Unit of doses can also include multiple pharmaceutical agents administered concomitantly or sequentially to the patient. For the remaining description of the systems and methods herein, the term “each dose” with be inclusive of “unit of doses” unless clearly indicated otherwise.

As used herein “administered” is inclusive of providing a dosage form to the patient by any route to introduce the pharmaceutical to the body of a patient including, for example, oral, parenteral, dermal, rectal, inhaled, injected, and the like. Administered is also inclusive of whether or not the pharmaceutical was successfully ingested, inhaled, etc. by the patient as determined by the route of administration.

In one embodiment, when the dosage regimen is completed and documented, the patient receives an incentive or reward for the successful completion of the dosage regimen. In another embodiment, the patient receives incremental incentives and/or rewards after administration of each dose, rather than at the completion of the regimen. In still yet another embodiment, an incentive and/or reward is given incrementally as well as at the completion of the dosage regimen. In the foregoing embodiments, the caregiver may be the one to receive an incentive and/or reward. Still further embodiments have both the patient and caregiver receiving the incentive and/or the reward.

The motivation and/or documenting devices can include information relating to the dosage regimen for the particular pharmaceutical agent. Such information can include, for example, the number of days of the regimen, the number of dosages, the relative times to administer the dosages, and the like. In one embodiment, the information is pre-printed on the device. Optionally, there is a location in or on the device for a doctor, pharmacist, patient and/or caregiver to provide the previously described information, provide a description of the dosage regimen, provide information about the patient, and/or other information.

Usually the documenting and motivation devices will be used by a caregiver, who is motivated to attain compliance with the dosing regimen for the patient that the caregiver is responsible for, as a means to motivate the patient. For example, the caregiver might be a parent and the patient a child, or the caregiver might be a nursing home staff member and the patient is a resident of the nursing home, etc. However, the devices could also be used for self-motivation by a patient who has enough self discipline to utilize such a device. Also, the documenting device could be used in any multiple dosage situation as a tracking mechanism, to remember which dosages have been administered, with or without the motivation aspect.

The documenting device is not particularly limited as long as it can be modified to memorialize the administration of the dosage units of the pharmaceutical agent or administration of the pharmaceutical agent at an appointed time in the regimen. Furthermore, the motivation device is not particularly limited as long as it can motivate, or is intended to motivate, the patient to adhere to the dosage regimen. Exemplary documenting and/or motivation devices can include cards; sheets; dry erase boards; magnetic boards; plates; games; videos; electronic data media, opto-electronic data media, and software therein; and the like organized or formatted in a way to provide motivation and/or facilitate tracking and documenting of the dosing regimen. For example, the device can be organized in a way to facilitate documenting of the dosing regimen, such as in the form of a calendar delineating days of the week, etc.

The devices can be fabricated from any variety of material, for example, paper, wood, fiber, cloth, plastic, metal, electronic formats, and the like. Specifically, cards or sheets prepared from paper can be used.

The documenting device can optionally further include, or be used in combination with, an indication device for indicating that a prescribed dosage unit was taken at the prescribed time. Exemplary indication devices include stickers or stamps (e.g., self-adhesive, moistenable, rubber stamp) that can be placed on the documenting device; a writing instrument (e.g., pen, pencil, marker, crayon, and the like) to indicate on the device when each dose has been administered; magnetic material; and the like.

Other exemplary indication devices can be integral to the documenting device itself. Such examples include the removal of a portion of the documenting device, such as a flap, perforated section, removal of a covering such as the coatings used for lottery scratch tickets, and the like. Still further, other indicating devices include the moving of a portion of the documenting device, such as a flap or window that can be opened by sliding a section of the documenting device.

In the embodiments where the indicating device is via the removal or moving of a portion of the documenting device (e.g., card or sheet), the action can reveal information, instruction, pictures, or codes on the documenting device that were previously unexposed. The information or instruction can be words of encouragement to the patient to continue the dosage regimen, description of the types of rewards that the patient can obtain after completing the regimen, and the like. Optionally, the code can be used by the patient or caregiver to contact the supplier or manufacturer of the pharmaceutical agent to receive further words of encouragement, description of the reward, aid in tracking the progress of the compliance with the regimen, and the like. The contact can occur via telephone, mail, email, interface with website prompt, other electronic formats, and the like.

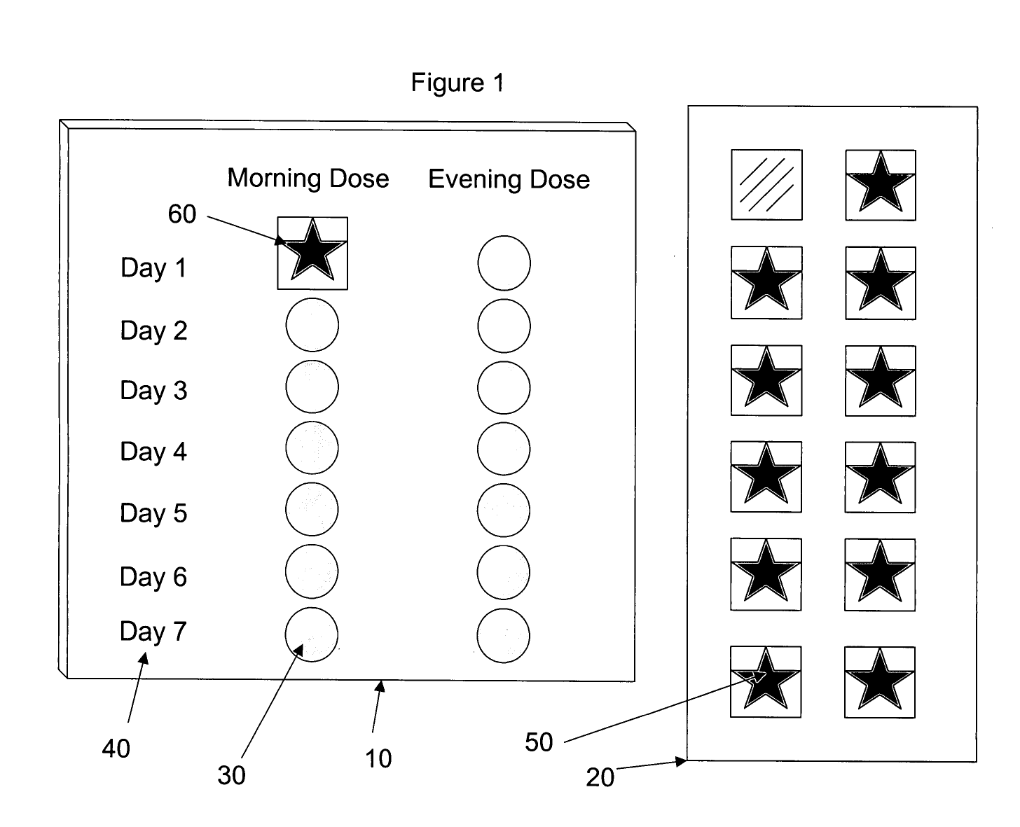

FIG. 1 provides an illustration of one embodiment of a compliance system. A documenting device is provided as a card (10) containing specified locations (30) for indicating when a particular dose has been administered. The indicating device includes self-adhesive stickers (50) that are provided as a sheet of stickers (20). Also provided on the card (10) are the days and relative times (40) according to the dosage regimen. A sticker placed on a dosage location (60) indicates that the morning dose of the pharmaceutical agent was administered. As discussed below, the card can be mailed in by the patient and redeemed for a reward.

In one embodiment, the completed documenting device, memorializing administration of all of the dosage units of the pharmaceutical agent according to the regimen, can be redeemed by the patient to obtain a reward or incentive. The completed documenting device can be redeemed by forwarding to a designated location via mail, electronically via facsimile, email or internet, returned to the doctor, pharmacy, or the location where the prescription is filled or dispensed, and the like. In another embodiment, the reward is provided at the time the prescription is obtained and can be provided to the patient by the caregiver at the completion of the dosage regimen. In another embodiment, the documenting device is not redeemed, but rather a code is provided by the documenting device which is used to obtain the reward.

The reward or incentive is not limited and can include, for example, toys, games, clothes (hats, scarves, t-shirts, etc.), jewelry (e.g., pins, ear rings, pendants, necklaces, bracelets, rings, etc.), candy, books, coupons, gift certificates, or combinations thereof. The type of reward can be chosen for the particular age group of the patient. For example, pediatric patients can choose from a particular set of toys or games, while geriatric patients or caregiver can choose from clothes, jewelry, books, coupons, gift certificates, and the like.

The reward or incentive can be co-marketed or cross-promoted with the latest television series, book series, movie promotions, video games, characters therein, etc. As such, the reward or incentive can change over time to keep up to date with the latest fashion and culture.

In another embodiment, the documenting device is via a central processing unit (CPU) which can contain or can access software to track the progress of a patient complying with a dosage regimen. For example, the patient or caregiver can call a designated phone number, send an email, interact with a web site, etc., any number of times to provide input as to the patient's adherence to the regimen. For example, the patient or caregiver can email each time a dose of the pharmaceutical agent is administered as prescribed. Such an input can be stored in a memory that can be accessed by the CPU. Other input can be provided such as patient's name, address, age, doctor's name, etc.

Also provided in the memory, or provided to the CPU as an input which can then be saved in a memory, is the particular dosage regimen for the patient. Once the CPU receives the correct number of inputs from the patient or caregiver as to administration of the dosage units over the course of the prescribed number of days of the dosage regimen, the patient or caregiver will be contacted via telephone, email, mail, and the like and informed of the successful completion of the dosage regimen. The patient or caregiver can also be provided a description of the type of reward that is available. Optionally, upon the CPU determining that the patient has completed the regimen, a reward is sent directly to the patient or caregiver.

In another embodiment, the documenting device or motivation device can include a video, video game, or other software linked to the dosing regimen as a way to motivate and/or reward the patient after successful administration of each dose or completed regimen. The video, video game, or other software can be provided to the patient in the form of a compact disc (CD), digital versatile disc (DVD) digital video recorder (DVR), or other data storage media made available with the prescription. Optionally, the device can be obtained via a weblink.

In another embodiment, the documenting and/or motivation device is in the form of a computer/video game, movie, or other form of entertainment or reward in which further levels or options are provided for the patient each time the administration of another dosage is indicated. Exemplary incremental rewards or incentives would be to structure a video or movie using “cliff hangers” where a break occurs at a crucial point in the story so that the patient will be motivated to take the next dosage to see what happens next in the story. For a video game, the next dosage could provide access to the next level in the game, add additional powers to the player, add additional lives, or provide some other desirable game benefit to the player who, in this case, is also the patient. Such a device could provide the caregiver with an access code so that only the caregiver can enter the data each time that the patient has been administered the medication dosage as a further incentive for the patient to take the medication properly.

The pharmaceutical agent, documenting/motivation device, and optionally indicating device, can be packaged together by the manufacturer for increased convenience. The documenting/motivation device can be provided on the container housing the pharmaceutical agent itself, provided by the manufacturer as a separate unit, provided by the manufacturer through the form of an instruction informing the patient or caregiver where and how to access the device (e.g. via a telephone number, website address, email address, etc.) or provided by the doctor or pharmacist at the time the prescription is filled or dispensed.

Also provided herein are methods to promote patient compliance with a pharmaceutical dosage regimen by providing to a patient a plurality of dosage units of a pharmaceutical agent according to a prescription; providing a dosage regimen for the pharmaceutical agent; documenting the administration of each dosage unit according to the dosage regimen; and optionally rewarding the patient and/or caregiver for the completion of the dosage regimen as evidenced by the documentation. As mentioned previously, the successful completion of the dosage regimen can be documented and redeemed by the patient or caregiver for a reward or provided directly through any known electronic format.

The foregoing system and method can be used in combination with any type of pharmaceutical agent and is not limited thereby. Classes of pharmaceutical agents that can be used in the systems and methods include, for example, antianxiety agents, antiarrhythmics, antibacterials, antibiotics, antidepressants, antidiabetics, antiepileptics, antifungal antihelminthics, antihistamines, antihypertensive agents, antiinfectives, antimalarials, antimicrobials, antimigraine agents, antimuscarinic agents, antineoplastic agents, antiprotozoal agents, antipsychotic agents, antiviral agents, β-blockers, chemotherapeutic agents, Cox-2 inhibitors, decongestants, diuretics, hypnotics, hypotensive agents, immunosuppresants, neuroleptics, opioid analgesics, peripheral vasodilators/vasoconstrictors, sedatives, and the like.

Due to the potential risk of antibiotic resistance, antibiotics are specifically contemplated herein. Antibiotics are also specifically mentioned here due to their common use in children and the need to motivate children to take their medication. Suitable antibiotics that can be used in the systems and methods include, for example, oral antibiotics of the following general classes: sulfa drugs; folic acid analogs; beta-lactams, including penicillins and cephalosporins; tetracyclines; macrolides; lincosamides; streptogramins; quinolones, including fluoroquinolones; polypeptides such as polymixins; aminocyclitols; glycopeptides; oxazolidinones; and the like. As used herein, “antibiotics” are inclusive of antibiotics, antibacterials, antimicrobials, antiinfectives, and the like.

Exemplary antibiotics include amoxicillin, the combination of amoxicillin and potassium clavulanate, ampicillin, the combination of ampicillin and sulbactam, atovaquone, azithromycin, carbenicillin, cefaclor, cefdinir, cefonicid, ceftibuten, cefotetan, cefpodoxime, ceftriaxone, cefuroxime, cephalexin, cephalothin, cephamycin, chlortetracycline, ciprofloxacin, clindamycin, clarithromycin, cycloserine, dalfopristin, dicloxacillin, doxycycline, erythomycin, levofloxacin, linezolid, moxifloxacin, mupirocin, oxytetracycline, penicillin, rifampin, quinupristin, the combination of dalfopristin and quinupristin, spectinomycin, sulfadiazine, sulfamethoxazole, sulfametrole, sulfamoxole, sulfalene, sulfanilamide, tetracycline, trimethoprim, the combination of trimethoprim and sulfamethoxazole, vancomycin, combinations thereof and the like.

A particular antibiotic can be trimethoprim, sulfamethoxazole, or a combination thereof. In one embodiment, the antibiotic combination can contain trimethoprim and sulfamethoxazole in a weight ratio of about 1:2 to about 1:6, specifically about 1:3 to about 1:5, and yet more specifically about 1:4 to about 1:5. For example, the combination can be dosed at about 8 milligrams/kilogram/day (mg/kg/day) of trimethoprim and 40 mg/kg/day of sulfamethoxazole.

In one embodiment, each dosage unit of the trimethoprim/sulfamethoxazole combination can contain trimethoprim present in an amount of about 10 to about 300 mg and sulfamethoxazole present in an amount of about 50 to about 1000 mg; more specifically trimethoprim present in an amount of about 20 to about 200 mg and sulfamethoxazole present in an amount of about 100 to about 900 mg; and yet more specifically trimethoprim present in an amount of about 30 to about 170 mg and sulfamethoxazole present in an amount of about 150 to about 850 mg. Specific dosage units comprise oral tablets and oral solutions or suspensions in an amount of about 1 to 20 milliliters, specifically about 3 to 15 milliliters, and more specifically about 5 to about 10 milliliters per dosage unit.

There is no limit to the particular dosage forms of the pharmaceutical agent. Exemplary dosage forms include tablets, capsules, powders, pellets, granules, liquids, suspensions, emulsions, etc. The dosage form can optionally be taste-masked. The mode of administration is also not limited and can include, for example, oral, parenteral, transdermal, buccal, dermal, rectal, inhaled, and the like.

The term “active agent” is a pharmaceutical agent used to treat a patient suffering from a particular disease or infection. The active agent includes solvates (including hydrates) of the free compound or pharmaceutically acceptable salt, crystalline and non-crystalline forms, all optical isomers, as well as various polymorphs of an active agent, either alone or in combination.

In one embodiment, the system is provided with a prescription of an antibiotic. The prescription can be prescribed to treat any variety of infection, including respiratory system diseases, otitis media, urinary tract infection, and the like.

In another embodiment, the system is provided with a prescription of trimethoprim and sulfamethoxazole either as oral solid or liquid dosage forms.

Also included herein are pharmaceutical kits comprising a combination of a plurality of dosage units of the pharmaceutical agent and a documenting and/or motivation device. The combination allows for the convenient prescription of the medication along with the system to provide or increase patient compliance. Such a kit would allow the doctor to prescribe the appropriate medication for the patient without having the doctor also provide the system. The pharmacist would find it convenient to fill the prescription as the system could be included in the packaging for the pharmaceutical agent.

The kits can contain the dosage units of the particular pharmaceutical agent in any format, for example blister packs, bottles, pouches, ampules, syringes, pre-filled syringes, droppers, pre-filled droppers, containers known in the art or easily ascertained by one of ordinary skill in the art, and the like. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, guidelines for administration, and/or guidelines for mixing the components.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The following examples further illustrate the invention but, of course, should not be construed as in any way limiting its scope.

### EXAMPLES

### Example 1

A 5-year-old male patient suffering from acute otitis media and having symptoms of Eustachian tube congestion is prescribed a 10 day regimen of the antibiotic combination trimethoprim/sulfamethoxazole as an oral suspension or tablet BID. A card indicating the days of the dosage regimen and a sheet of stickers is provided with the prescription. The patient or patient's caregiver adheres a sticker to the card for each successful administration of the dosage units of the antibiotic combination according to the regimen. Upon successful completion of the dosage regimen, the completed card is mailed to the manufacturer and redeemed for a prize.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system to promote patient compliance with a pharmaceutical dosage regimen, comprising:
a documenting device to document administration to a patient of each dosage of a pharmaceutical agent according to a dosage regimen, or
a motivation device to motivate a patient to adhere to a dosage regimen for a pharmaceutical agent, wherein the motivation device is optionally also a documenting device; and
optionally further comprising an indicating device;
wherein the patient or caregiver is rewarded for the patient completing the dosage regimen.

2.-3. (canceled)

4. The system of claim 1, wherein the system further comprises a plurality of dosage units of the pharmaceutical agent.

5. The system of claim 1, wherein the system is provided with a filled or dispensed prescription of the pharmaceutical agent.

6. (canceled)

7. The system of claim 1, wherein the documenting device is modified to indicate the administration of each dosage according to the dosage regimen.

8. The system of claim 7, wherein the documenting device is modified by marking the device, attachment of stamps or stickers to the device, removing portions of the device, moving portions of the device, or a combination thereof.

9. (canceled)

10. The system of claim 1, wherein the indicating device is a stamp, a sticker, a removable portion of the documenting device, a movable portion of the documenting device, or a combination thereof.

11. The system of claim 21, wherein the motivation device is a game, a video, a video game, or software available on a compact disc, a digital versatile disc, or a digital video recorder disc; and wherein the patient is motivated with increasing usage of, or access to, a game or entertainment with increasing or continued compliance with the dosing regimen.

12.-14. (canceled)

15. The system of claim 1, wherein the patient is rewarded for completing the dosage regimen of the pharmaceutical agent as evidenced by a completed documenting device.

16. The system of claim 1, wherein the patient is rewarded with a toy, a game, clothes, jewelry, candy, a book, a coupon, a gift certificate, or a combination thereof.

17. The system of claim 1, wherein the documenting device is a card and the indicating device comprises a plurality of stickers.

18. The system of claim 1, wherein the pharmaceutical agent comprises antianxiety agents, antiarrhythmics, antibacterials, antibiotics, antidepressants, antidiabetics, antiepileptics, antifungal antihelminthics, antihistamines, antihypertensive agents, antiinfectives, antimalarials, antimicrobials, antimigraine agents, antimuscarinic agents, antineoplastic agents, antiprotozoal agents, antipsychotic agents, antiviral agents, β-blockers, chemotherapeutic agents, Cox-2 inhibitors, decongestants, diuretics, hypnotics, hypotensive agents, immunosuppresants, neuroleptics, opioid analgesics, peripheral vasodilators/vasoconstrictors, sedatives, or a pharmaceutically acceptable combination thereof.

19. (canceled)

20. The system of claim 18, wherein the pharmaceutical agent comprises trimethoprim, sulfamethoxazole, or a combination thereof.

21. A method to promote patient compliance with a pharmaceutical dosage regimen, comprising:
using a documenting device to document the administration to a patient of each dosage of a pharmaceutical agent according to a dosage regimen, or
providing a motivation device to motivate a patient to adhere to a dosage regimen for the pharmaceutical agent, wherein the motivation device is optionally also a documenting device;
optionally using an indicating device; and
rewarding the patient or caregiver for the patient completing the dosage regimen as evidenced by the documentation.

22.-23. (canceled)

24. The method of claim 21, further comprising providing to a patient or caregiver a plurality of dosage units of the pharmaceutical agent.

25. The method of claim 21, wherein the documenting is provided by modifying a card for each dosage administered according to the regimen;
wherein the modifying is by marking, attachment of stamps or stickers, removing portions of the card, moving portions of the card, or a combination thereof.

26. The method of claim 21, wherein the documenting device is a card used in combination with an indicating device, and wherein the indicating device comprises a plurality of stickers;
wherein a sticker is placed on the card to indicate the administration of a dosage; and
wherein the rewarding is provided by redeeming the card documenting the patient's completion of the dosage regimen.

27. The method of claim 21, wherein the motivation and/or documenting device is provided with a filled or dispensed prescription of the pharmaceutical agent.

28. The method of claim 21, wherein the motivation and/or documenting device is a game, a video, a video game, or software available on a compact disc, a digital versatile disc, or a digital video recorder disc.

29.-31. (canceled)

32. The method of claim 21, wherein the patient is rewarded with a toy, a game, clothes, jewelry, candy, a book, a coupon, a gift certificate, or a combination thereof.

33.-35. (canceled)

36. A kit to promote patient compliance with a pharmaceutical dosage regimen, comprising:
a plurality of dosage units comprising a pharmaceutical agent;
instructions of a dosage regimen for the pharmaceutical agent; and
the system of claim 1.

37.-40. (canceled)
